(19) United States
(12) Patent Application Publication
(10) Pub. No.: US 2026/0157895 A1
(43) Pub. Date: Jun. 11, 2026

(54) HEATED GURNEY FOR MAINTAING PATIENT BODY TEMPERATURE

(71) Applicant: Tyler Melson, Barboursville, WV (US)

(72) Inventor: Tyler Melson, Barboursville, WV (US)

(21) Appl. No.: 18/969,482

(22) Filed: Dec. 5, 2024

(51) Int. Cl. A61G 1/04; A61G 1/02

(52) U.S. Cl. CPC A61G 1/04; A61F 7/007; A61G 1/02; A61G 2210/90

(57) ABSTRACT

A patient transport apparatus for warming a patient during emergency transport includes a gurney with a frame and a platform. The platform is mounted atop the frame and is elongated between a head end and a foot end. The platform has a pair of lateral sides, each of which extends between the head end and the foot end. A pad is mounted atop the platform of the gurney. The pad has an upper surface such that the pad may support the patient above the gurney. A heater is embedded within the pad.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (F) STATEMENT REGARDING PRIOR DISCLOSURES BY THE INVENTOR OR JOINT INVENTOR

Not Applicable

### (G) BACKGROUND OF THE INVENTION

### (1) Field of the Invention

The disclosure relates to patient transport devices and more particularly pertains to a new patient transport device for warming a patient during emergency transport.

### (2) Description of Related Art Including Information Disclosed Under 37 CFR 1.97 and 1.98

Circulatory shock is a significant cause of death in individuals who have experienced traumatic injuries. In particular, hypovolemic shock, which occurs due to significant blood loss, is a common and life-threatening danger. It is difficult to transport sufferers of circulatory shock quickly enough to areas where professional assistance and adequate blood sources for transfusions are available. Maintaining a patient's body temperature is one way to slow the fatal effects of circulatory shock.

The prior art describes many devices which heat or insulate a patient in order to maintain the patient's body temperature at adequate levels. However, these typically do not provide a significant heat source that is available to use during the process of transporting the patient. The prior art fails to describe such an apparatus in which a heat source is incorporated into a gurney and may be powered by accessing an ambulance's electrical system.

### (H) BRIEF SUMMARY OF THE INVENTION

An embodiment of the disclosure meets the needs presented above by generally comprising a gurney with a frame and a platform. The platform is mounted atop the frame and is elongated between a head end and a foot end. The platform has a pair of lateral sides, each of which extends between the head end and the foot end. A pad is mounted atop the platform of the gurney. The pad has an upper surface such that the pad may support a patient above the gurney. A heater is embedded within the pad.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (I) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front in-use view of a patient transport apparatus according to an embodiment of the disclosure.

FIG. 2 is a top view of an embodiment of the disclosure.

FIG. 3 is a cross-sectional view of an embodiment of the disclosure taken from Line 3-3 in FIG. 2.

### (J) DETAILED DESCRIPTION OF THE INVENTION

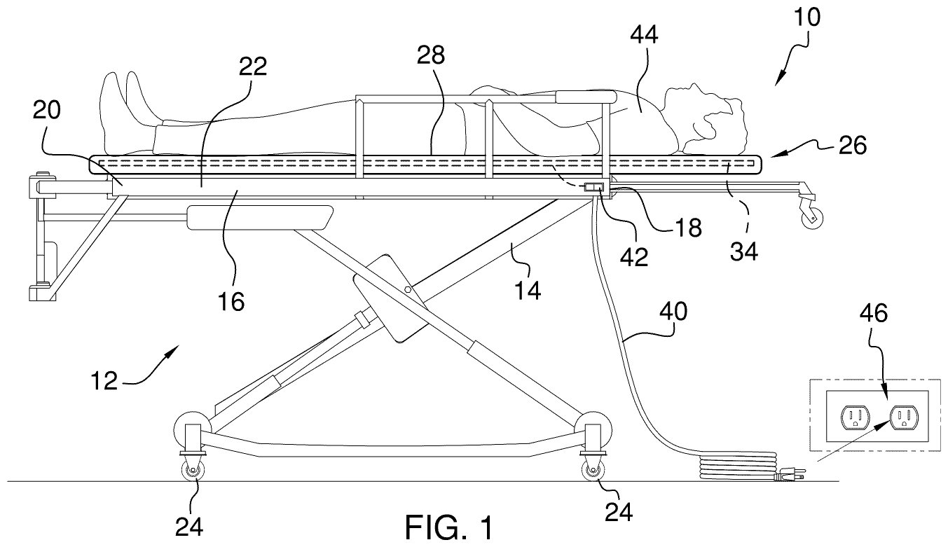

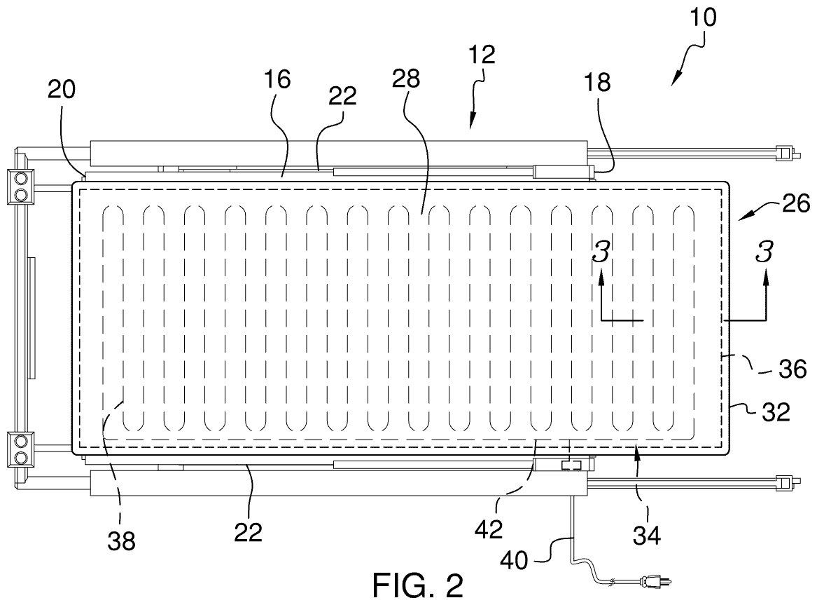

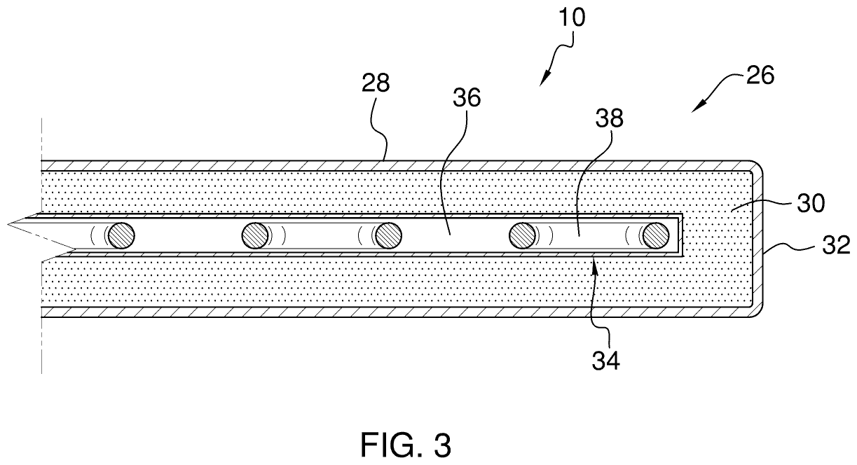

With reference now to the drawings, and in particular to FIGS. 1 through 3 thereof, a new patient transport device embodying the principles and concepts of an embodiment of the disclosure and generally designated by the reference numeral 10 will be described.

As best illustrated in FIGS. 1 through 3, the patient transport apparatus 10 generally comprises a gurney 12 with a frame 14 and a platform 16, wherein the platform 16 is mounted atop the frame 14. The platform 16 is elongated between a head end 18 and a foot end 20 and has a pair of lateral sides 22 extending between the head end 18 and the foot end 20. The gurney 12 further comprises a plurality of wheels 24 mounted to a bottom of the frame 14.

A pad 26 is mounted atop the platform 16 of the gurney 12. The pad 26 has an upper surface 28 wherein the pad 26 is configured to support a patient 44. The pad 26 comprises a cushion 30 formed of a deformable material such that the cushion 30 deforms when the pad 26 supports the patient 44. The deformable material may be a rubber foam, a plastic foam, a cotton filling, or the like. The pad 26 further comprises a casing 32 which surrounds the cushion 30. The casing 32 may be fluid impermeable such that bodily fluids, water, and other fluids do not penetrate and soak the cushion 30.

A heater 34 is embedded within the cushion 30 of the pad 26 and comprises a housing 36 and a resistive heating element 38. The resistive heating element 38 is mounted in the housing 36. An electrical cord 40 is electrically coupled to the resistive heating element 38 of the heater 34. The electrical cord 40 extends from the resistive heating element 38 through the platform 16 of the gurney 12. The electrical cord 40 is configured to electrically couple to an ambulance electrical system 46 which may include an electrical inverter.

A switch 42 is operatively coupled to the heater 34. The switch 42 is selectively actuatable to alternately electrically decouple and electrically couple the electrical cord 40 to the resistive heating element 38. The switch 42 is mounted to the gurney 12 and is positioned on an associated lateral side 22 of the pair of lateral sides 22 of the platform 16 of the gurney 12. The switch 42 is further positioned proximate to the head end 18 of the platform 16 of the gurney 12.

In use, the patient 44 is placed on the upper surface 28 of the pad 26, and the gurney 12 is moved to transport the patient 44. The electrical cord 40 may couple via a receptacle or the like to the ambulance electrical system 46. The electrical cord 40 may also couple to another external power supply such as a utility power supply delivered to a structure's electrical system. When the electrical cord 40 is electrically coupled to a sufficient power supply, the switch 42 may be actuated to couple the electrical cord 40 to the resistive heating element 38 of the heater 34 which begins generating heat. The heat is transferred through the pad 26 to the patient 44 to raise the body temperature of the patient 44 which may stave off circulatory shock in the patient 44.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A patient transport apparatus for warming a patient during transport, the patient transport apparatus comprising:
a gurney, the gurney comprising a frame and a platform, the platform being mounted atop the frame, the platform being elongated between a head end and a foot end, the platform having a pair of lateral sides extending between the head end and the foot end;
a pad mounted atop the platform of the gurney, the pad having an upper surface wherein the pad is configured to support the patient; and
a heater coupled to the pad, the heater being embedded within the pad.

2. The patient transport apparatus of claim 1, wherein the gurney further comprises a plurality of wheels mounted to a bottom of the frame.

3. The patient transport apparatus of claim 1, wherein the pad comprises a cushion having a deformable material wherein the cushion is configured to deform when the pad supports the patient.

4. The patient transport apparatus of claim 3, wherein the pad further comprises a casing which surrounds the cushion.

5. The patient transport apparatus of claim 1, wherein the heater comprises a housing and a resistive heating element, the resistive heating element being mounted in the housing.

6. The patient transport apparatus of claim 5, further comprising an electrical cord electrically coupled to the resistive heating element of the heater, the electrical cord extending out of the gurney, the electrical cord being configured to electrically couple to an ambulance electrical system.

7. The patient transport apparatus of claim 6, further comprising a switch operatively coupled to the heater, the switch being selectively actuatable to alternately electrically decouple and electrically couple the electrical cord to the resistive heating element.

8. The patient transport apparatus of claim 7, wherein the switch is mounted to the gurney, the switch being positioned on an associated lateral side of the pair of lateral sides of the platform of the gurney, the switch being positioned proximate to the head end of the platform of the gurney.

9. A patient transport apparatus for warming a patient during transport, the patient transport apparatus comprising:
a gurney, the gurney comprising a frame and a platform, the platform being mounted atop the frame, the platform being elongated between a head end and a foot end, the platform having a pair of lateral sides extending between the head end and the foot end, the gurney further comprising a plurality of wheels mounted to a bottom of the frame;
a pad mounted atop the platform of the gurney, the pad having an upper surface wherein the pad is configured to support the patient, the pad comprising a cushion having a deformable material wherein the cushion is configured to deform when the pad supports the patient, the pad further comprising a casing which surrounds the cushion;
a heater coupled to the pad, the heater being embedded within the cushion of the pad, the heater comprising a housing and a resistive heating element, the resistive heating element being mounted in the housing;
an electrical cord electrically coupled to the resistive heating element of the heater, the electrical cord extending out of the gurney, the electrical cord being configured to electrically couple to an ambulance electrical system; and
a switch operatively coupled to the heater, the switch being selectively actuatable to alternately electrically decouple and electrically couple the electrical cord to the resistive heating element, the switch being mounted to the gurney, the switch being positioned on an associated lateral side of the pair of lateral sides of the platform of the gurney, the switch being positioned proximate to the head end of the platform of the gurney.
